(19) United States
(12) Patent Application Publication
(10) Pub. No.: US 2012/0016427 A1
(43) Pub. Date: Jan. 19, 2012

(54) OSTEOSYNTHESIS DEVICE

(75) Inventors: Eric Stindel, Locamaria Plouzane (FR); Christian Lefevre, Brest (FR)

(73) Assignee: OSTESYS, Plouzane (FR)

(21) Appl. No.: 13/146,826

(22) PCT Filed: Jan. 28, 2010
(86) PCT No.: PCT/EP2010/051035

§ 371 Date: Oct. 10, 2011

(30) Foreign Application Priority Data
Jan. 28, 2009 (FR) FR0950501

(51) Int. Cl. A61B 17/56

(52) U.S. Cl. 606/86 R

(57) ABSTRACT

An osteosynthesis device is provided. The device includes a first screw for attaching a first holding base to a first section of a bone, a second screw for attaching a second holding base to a second section of the bone, and a rigid linking element mutually connecting the first and second bases. Either the first screw or the first base has a cavity for receiving a bracket of an associated position-marking device.

### CROSS-REFERENCE TO RELATED APPLICATIONS

This Application is a Section 371 National Stage Application of International Application No. PCT/EP2010/051035, filed Jan. 28, 2010 and published as WO 2010/086390 A1 on Aug. 5, 2010, not in English.

### STATEMENT REGARDING FEDERALLY SPONSORED RESEARCH OR DEVELOPMENT

None.

### THE NAMES OF PARTIES TO A JOINT RESEARCH AGREEMENT

None.

### FIELD OF THE DISCLOSURE

The disclosure pertains to osteosynthesis devices.

When a person has a bone structure with deformed legs, such that the knees are excessively or insufficiently apart in the standing position, then the weight of the body, which is unequally distributed at the knee, can cause arthrosis. To remedy this problem, the above-mentioned deformation needs to be eliminated as far as possible by the by changing the direction of extension (re-tilting or axial correction) of the tibia to bring it into a predetermined angular range relatively to the femur. The optimal range is considered to be in the range of 183 degrees to 186 degrees.

The angle between these two directions of extension, called the HKA angle, is defined on the basis of three joint centers of the thigh, i.e. an intermediate common center at knee level, the other two centers being respectively situated at the hip and at the ankle

### BACKGROUND OF THE DISCLOSURE

To re-tilt the direction of extension the tibia, an osteotomy is performed by lateral incision in the upper part of the tibia, and then the lower section of the tibia can tilted to the desired angle of alignment correction. Then, the two sections of the tibia are blocked by a plate screwed on to these two sections which will thereafter be knitted together by bone growth. Since the tibia is thus re-tilted laterally, the ankle joint center is shifted laterally so as to be better aligned with the upper reference line constituted by the respective centers of rotation of the hip and knee.

During the operation, it is difficult to measure the value of the HKA because the above joint centers are purely geometrical, i.e. they are not visible elements. The classic solution therefore is to use position markers or trackers, detectable by a computer or by a navigation station which will assist the surgeon and thus increase the precision of the surgery. Classically, these markers or trackers are screwed temporarily into the femur and into two sections of the tibia respectively, specifically the tibial epiphysis above the planned cutting level, and the tibia diaphysis just beneath the cutting level. The 3D information items obtained by means of the position trackers devices make it possible to adjust the HKA angle. This is done for the front plane of the opening made in the tibia and also for the sagittal plane (the slope) as well as for the transversal plane (rotation).

Once the operation is completed, an osteosynthesis plate is screwed on to the two sections to hold them in their relative position and then the position trackers are unscrewed.

One drawback of such a process is that the position trackers and their attachment screws take up space and are therefore inconvenient during the osteotomy. Furthermore, these screws harm the integrity of the bone, and do so specifically in an area that will additionally receive the attachment screws of the osteosynthesis plate.

### SUMMARY

An exemplary embodiment of the present invention seeks to propose a solution making it possible to at least limit the severity of at least one of the above-mentioned two problems.

An embodiment of the invention pertains to an osteosynthesis device comprising:

first attachment means for attaching a first holding base to a first section of a bone,

second attachment means for attaching a second holding base to a second section of said bone, and

rigid linking means disposed so as to mutually connect the first and second bases.

According to an embodiment of the invention, at least one of the first attachment means and the first base comprise first means for receiving at least one first bracket of an associated position-tracking device.

Thus, an embodiment of the invention proposes a functionally staged structure, i.e. the bone will be coupled to the first attachment means and hence to the first base and one of these two types of elements will serve as a receiving interface for the bracket of the position-tracking device.

In the prior art on the contrary, the bone rests directly on the first attachment means as well as the bracket of the tracking device which are therefore functionally side by side and not functionally stacked on one another.

An embodiment of the invention therefore consists of the transfer, to the first base and/or to the associated first attachment means, of the function of receiving the bracket of the tracking device, previously provided on the bone by screwing into this bone.

This transfer has two beneficial effects.

First of all, since the first attachment means can be easily made out of a material that is harder than bone, for example metal or ceramic, small-sized coupling surfaces are enough to ensure the desired mechanical coupling on the bracket, without any risk of wrenching or of play. The coupling elements according to an embodiment of the invention can thus be miniaturized. On the contrary, in the prior art, bone requires relatively long screws of a fairly big diameter to distribute the stresses and thus prevent any swivel which would be a source of lack of precision of the identified position and would damage the bone.

Finally, the integrity of the bone is now free of any harm caused by the screws or equivalent means for attaching brackets of tracking devices.

As explained further below, the first and second bases may or may not be distinct elements assembled by rigid linking means, having the function of an osteosynthesis plate. Furthermore, all or part of these three elements can be mounted at the outset on the bone or else coupled with attachment means after they have been mounted on the bone.

According to one particular embodiment, the second attachment means comprise second means of receiving at least one second bracket of a second position-tracking device.

The concept of an embodiment of the invention is thus applied to the entire device.

According to one particular aspect of an embodiment of the invention, at least the first receiving means are disposed so that the bracket of the associated position-tracking device is carried removably.

Thus, the patient's discomfort is limited.

According to one embodiment, the first attachment means include a first headed attachment screw comprising first receiving means, disposed so as to receive and get coupled to the bracket of the first position-tracking device.

In such a case, it can be planned that the first receiving means will constitute a tip relief feature of the head of the first attachment screw.

It may for example be a tip cavity with a non-circular cross section, for example with a lateral surface with faces to block any rotation of the tracking device. This device can then be held in the coupling position, for example by an elastic strip, a clip or again by the underneath of a secondary screw head that gets screwed into a tapped hole made at the bottom of the tip cavity.

In a dual manner, the attachment screw head may have a coupling neck for coupling with a cavity of the position-tracking device.

It can also be planned that the attachment screw head will comprise an external thread for the screwing thereon of a tapped hole made in the tracking device, it being thus possible to envisage a dual assembly. In such a case, the attachment screw head can have a diameter, or overall dimensions if it is not circular, of any unspecified value relative to the diameter of the screw body, since the screw head then functionally constitutes a specific section of a screw body. If the functional part, the tracking part, of the tracking device is on the screw axis of the above screw, the final angular position may be any unspecified position. If not, an angular indexing tracker is used to stop the screwing in the desired angular position so that the functional tracking part is in the desired azimuth direction relatively to the attachment screw head.

The tip relief feature can be disposed to receive a clamping screw.

The attachment screw is thus a two-staged screw, i.e. the clamping screw is a detachable head of the attachment screw.

In one particular embodiment, the first bracket belongs to the osteosynthesis device and comprises a point of anchoring to the associated bone section.

Thus a primary anchoring, associated with a secondary anchoring, is constituted. The secondary anchoring can easily be planned to be at a certain distance from the primary anchoring, i.e. the tracking device is then borne by two legs at a distance from each other, therefore having high stability against tilt. The secondary anchoring can thus be limited to being an anti-tilt support. However, it can have a certain degree of penetration into the bone, so as to thus prevent any rotation around the primary anchoring.

The second base can comprise a toe disposed so as to slide between a surface zone of the second bone section and a lower facing surface belonging to the attaching element.

The first base can thus be easily added on without any need to dismantle the first receiving means, thus preventing any risk of introducing error into a setting made with these first receiving means.

Furthermore, one of the bases may comprise an aperture for setting the position of the associated attachment means.

According to one particular embodiment, the linking means comprise a strap comprising first and second coupling means for coupling respectively with the first and second bases, the first coupling offering a degree of freedom in rotation between the first base and the strap to set an angular value of relative rotation between the first and second bases, and therefore also of the bone sections, and the second coupling means offering a degree of freedom in longitudinal translation to set a value of longitudinal distance between the first and second bases.

It can be planned that the second base will comprise a rough coupling surface to cooperate with a homologous surface of the second coupling means of the strap, in which a longitudinal aperture is made for the passage of a second headed attachment screw designed to mutually immobilize said surfaces by mutual pressure.

A simple screwing operation thus ensures the blocking of the first and second bases in their desired relative positions.

The rough coupling surface may for example have rows of grooves/ridges or again a matrix of localized relief features. The corresponding surface may have complementary relief features, i.e. in fact the same kind of relief features. It may however be planned that only one of the surfaces will have such relief features and that the other surface will be the surface of a layer of material softer than the material of the surface with relief features.

The first and second bases are advantageously separate elements comprising supporting surfaces for respective distractors.

Surgery is thus facilitated.

The device according to an embodiment of the invention makes it possible especially to perform a cutting operation according to the method comprising steps for:
a implanting, on two respective sections of a reclining element, two screws for attaching a holding plate, the screws being also provided for the bracket of respective position-tracking devices,
b mounting the brackets of position-tracking devices, bearing said position-tracking devices,
c performing a cutting operation between the first and second sections,
d performing an operation of distracting the first and second sections in holding them in a desired final position,
e mounting a holding plate, or osteotomy plate, on the attachment screws and on the bracket, and
f performing a clamping of the holding plate of the attaching and holding screws.

After the cutting operation, it may planned to insert a wedge between the first and second sections and then to withdraw all tools as well as the brackets bearing the position-tracking devices. It will be noted that the device can be used for any cutting operation in a material to which an attachment is made, for example by screwing.

In other embodiments indicated here below, it is possible to mount initially the first and second bases because the mechanism initially leaves the desired degree or degrees of freedom between the first and second bases. It can thus be planned that the brackets will be implanted directly on the plate, preferably in a removable way.

The above steps are associated with steps of operational assistance, in that the positions of the position-tracking devices are detected by a work station which, on the basis of the positional information received, makes a real-time computation of the position of the second bone section relatively to a direction of reference corresponding to the direction of the femur. The image of the second section, or at least its direction of extension (3D orientation) and its 3D position are displayed on a screen, possibly with numerical information, by the current angle HKA or its difference relative to a set value of angle sought for the operation. Information for guiding the surgeon may furthermore be provided on the screen or by voice synthesis, in order to thus set the desired correction angle. It can furthermore be planned that one or more actuators, controlled by the station, will perform certain movements to shift the second bone section, i.e. there is then a feedback loop system, the position-tracking devices enabling feedback control over the above actions.

In one promising embodiment, the device has ball-and-socket means comprising a pair of first and second cooperating joint means, any one of these means being associated with the first base and the other one being associated with the linking means, the first joint means have a volume template of overall space requirement smaller than a housing volume template, presented by the second joint means, so as to be housed therein with clearance and a possibility of mutual rotation along three axes, in a state of rest of the ball-and-socket means,

and comprising locking means disposed so as to make the joint means pass into a locked state by modification of one of said volume templates so as to eliminate said clearance in at least two distinct zones of each of the volume templates.

The joint means associated with the first base can be associated with it in various ways, i.e. integrated with it or mounted on it or again associated with it through the first attachment means, for example in being mounted on an attachment screw belonging to the first attachment means.

Since three degrees of freedom in rotation are available, the two bases and therefore the two bone sections can be oriented independently. The mounting of the bases on the respective bone sections thus does away with the need for any precise positioning in orientation, thus greatly simplifying this mounting. It can furthermore be planned to have a set of bridging straps between the first and second bases in order to cover all possible angles of opening and a navigation system will choose the appropriate strap.

The adjusted locking position is obtained by a pressure force exerted by the joint means which get deformed to be supported by the other, this pressure force creating a force for holding in position through a coefficient of friction of the two surfaces in contact or through a coupling of opposite relief features. In the latter case, one of the surfaces in contact may thus have a certain roughness, or even a regular pattern of ridges, with a certain hardness and the opposite surface may be smooth with a lower degree of hardness so that the opposite relief features get imprinted therein and block any subsequent rotation.

It will be noted that the above ball-and-socket means can thus be used in a framework other than that described in the present application, i.e. a) in a non-medical field or again b) with classic bases of first attachment means, i.e. means unsuited to receiving a first bracket of an associated position-tracking device.

In a preferred embodiment, the template of overall space requirement corresponds to a slice of a substantially spherical volume having a first determined radius and the housing template corresponds to a slice of a substantially spherical volume having a second determined radius, the locking means being disposed to deform one of the joint means so as to at least locally modify the radius until equality is attained with the radius of the other of the joint means to thus set up a contact bringing about a passage to the locked state.

In such a case, it can be planned that the first joint means will comprise a ring, with a radially external surface defining said template of overall space requirement, and a radially internal surface mounted so as to be sliding on a sloping surface of an expansion neck, that is functionally in the form of a truncated cone, belonging to the locking means, this ring being associated with an element providing thrust in said sliding operation and holding the assembly in the final locking position.

The housing in the form of a spherical slice thus has a profile limited by two “C”-shaped curves open towards each other and the ring similarly has an external profile limited by two “C”-shaped curves open towards each other. The diameter of the ring thus increases to reach the diameter of the housing. The contact of the two surfaces coupling with each other will be total if they are both perfectly spherical and precisely if the first radius of sphericity corresponds to the expanded state of the ring, i.e. it is equal to the second radius of sphericity. The two respective centers of sphericity will then coincide. If these centers are in the housing, the top and bottom pairs of the horns of the C-shaped curves bounding it demarcate the narrowed portions that imprison the expanded ring. If not, if the housing is in the shape of a bowl, the ring will have to be associated with means for holding in the housing, for example by means of said neck.

In one particular embodiment, the expansion neck is constituted by a head of said first attachment screw of the first base. The screw is thus bi-functional, making it possible to omit a specific element.

Advantageously, the thrusting and holding element is a screw comprising said first receiving means for receiving at least one first bracket of an associated position-tracking device.

In a first alternative embodiment, the locking means comprise a sliding element laid out so as to slide in an interstice volume offered by said clearance, until it comes into a position of being supported on both the first joint means and the second joint means.

To get housed by an increase in the clearance on one side, the sliding element thus moves away the associated facing zones belonging respectively to the first joint means and second joint means and, on an opposite side, it therefore causes a reduction of the local clearance which brings into contact the zones of these means that are mutually facing each other and will therefore serve as a support to finally block said increase in clearance and therefore block the advance, in the interstice volume, of the sliding element constituting an ankle for locking by friction.

The sliding element can be guided and held in a groove of the first and second joint means, i.e. it is integrated in a moveable way in these joint means to modify the shape of the template according to said deformation of coupling with the other joint means. The sliding element is advantageously a screw, the threads of which provide for holding in the locked position.

In a second alternative embodiment, the template of overall space requirement offers a spherical portion and the housing template offers an associated truncated-cone portion.

The mutual contact is therefore ensured in a circle which provides a well distributed contact having a high torque of resistance to pivoting when the lever arm is equal to the diameter of this circle.

In a second embodiment proposed as a variant, the template of overall space requirement of the first joint means is compatible with a first housing template presented by a first section of said housing belonging to said second joint means and is incompatible with a second housing template presented by a second housing section, and the locking means are disposed so as to push back the first joint means, from the first section to the second section.

Here again, it may be planned that the surfaces to be coupled will be spherical but contrary to the case of the deformable ring with variable diameter, it is a simple translation that provides for the coupling.

An embodiment of the invention also pertains to a computer program product, downloadable from a communications network and/or stored on a computer-readable medium and/or executable by a microprocessor, characterized by the fact that it comprises program code instructions to execute at least some of the steps of the above method which is executed on a computer.

An information system can thus help in the desired navigation.

### BRIEF DESCRIPTION OF THE DRAWINGS

The invention will be understood more clearly from the following description of four embodiments of an osteosynthesis device, presented by way of a non-exhaustive illustration, with reference to the appended drawings, of which:

FIG. 1 is formed by FIGS. 1A, 1B, 1C and 1D each representing a longitudinal section of the knee joint in a sequence of operational steps in which an upper zone of the tibia is sectioned laterally to realign the rest of the tibia,

FIG. 2 is formed by the FIGS. 2A and 2B which are lateral views in two different directions of the tibia of FIG. 1 once operated, the two sections of the tibia being then mutually blocked in a desired position by an osteosynthesis device according to an embodiment of the invention,

FIG. 3 is a plane view of the first embodiment of the device comprising an osteosynthesis plate which is a monoblock device,

FIG. 4 is an exploded view in perspective of a mounting of a bracket of a device for tracking the position of a bone section,

FIG. 5 is a schematic sectional view of a bone bearing the element of FIG. 4,

FIG. 6 shows two screws for attaching two bases of FIG. 3,

FIG. 7 is an exploded view of an attachment screw head used as a receptacle for a bracket of a position-tracking device,

FIG. 8 is a view in exploded perspective of an attachment screw and bracket that are associated,

FIG. 9 corresponds to FIG. 8, the elements being partially assembled,

FIG. 10 is a view in perspective of the attachment screw head and the receptacle for the bracket,

FIG. 11 represents the elements of FIGS. 8 and 9 mounted on the tibia,

FIG. 12 represents the osteosynthesis plate positioned on the tibia,

FIG. 13 corresponds to FIG. 12 with, in addition, attachment screws that are to be positioned,

FIG. 14 corresponds to FIG. 13 after the dismantling of the brackets of the position-tracking device,

FIG. 15 is a view in perspective of the second embodiment of the device, with an osteosynthesis plate that is a variant of the first embodiment,

FIG. 16 is a view of four attachment screws of the device of FIG. 15,

FIG. 17 is a view in exploded perspective showing the heads of the attachment screws of FIG. 16 as well as said brackets of a position-tracking device,

FIG. 18 shows the result of the osteotomy,

FIG. 19 is the view in exploded perspective showing the attaching of said brackets on the basis of the configuration of FIG. 15,

FIG. 20 shows the installation of four clamping screws,

FIG. 21 is a plane view of a third embodiment, with an osteosynthesis plate with separate bases,

FIG. 22 shows a grip for temporarily holding the osteosynthesis plate of FIG. 21,

FIG. 23 is an exploded view of the elements of FIG. 22,

FIG. 24 shows the positioning of four attachment screws,

FIG. 25 illustrates the mounting of two said brackets and their attachment by thumb wheels,

FIG. 26 is a plane view showing two distractors resting on bases of the osteosynthesis plate,

FIG. 27 shows the installation of four clamping screws on the above plate,

FIG. 28 is a view in exploded perspective showing an attachment screw head which receives a clamping screw,

FIG. 29 is a plane view showing the osteosynthesis plate in the definitive mounting state,

FIG. 30 is a flow chart showing the operational steps,

FIG. 31 is an axial view in section of a ball-and-socket-type joint according to the fourth embodiment, linking said osteosynthesis plate with an attachment screw in a bone, and

FIG. 32 is an overall view in exploded perspective of the elements of FIG. 31.

### DETAILED DESCRIPTION OF ILLUSTRATIVE EMBODIMENTS

### 1. First Embodiment

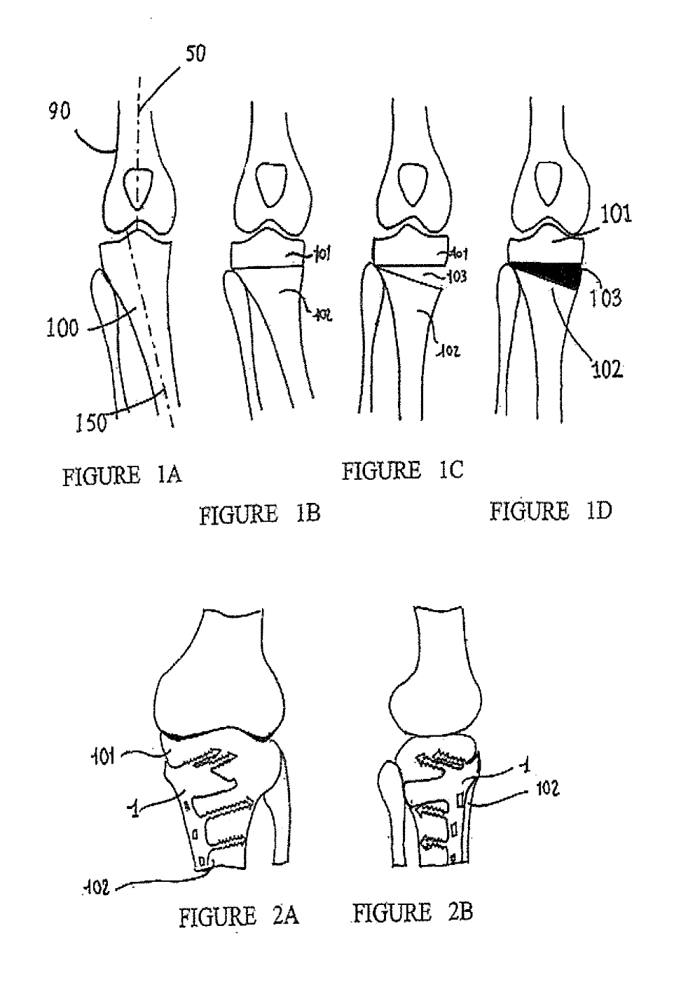

FIGS. 1A to 1D schematically illustrate the progress of an osteotomy operation for opening a bone 100 which in this case is a tibia, i.e. an elongated bone. Clearly, the bone 100 could be any other bone whose shape has to be modified by incision.

In FIG. 1A, a femur 90 has a direction of extension 50 which represents precisely a straight line linking a geometrical center of rotation of the upper end of the femur 90, situated in the hip, to a geometrical center of rotation at the knee. The tibia 100 has a direction of extension 150 which corresponds to a straight line linking its geometrical center of rotation of the knee with its geometrical center of rotation at the ankle It can be seen that the directions 50, 150 have a relative obliqueness which is excessive. It is therefore necessary to make the tibia 100 pivot in a clockwise direction in order to reduce this angle.

FIG. 1B shows that an osteotomy is made on the tibia 100, i.e. that the tibia is cut into two sections 101, 102 in its upper part just below the knee joint.

FIG. 1C shows that the lower section 102 has undergone the desired pivoting to bring the angle of divergence between the directions 50, 150 into the desired range of 183 to 186 degrees. Clearly, an embodiment of the invention is not limited to the choice of such a range. The facing slices of the two sections 101, 102 therefore extend in surfaces inclined relatively to each other according to the pivoting angle that has been applied.

FIG. 1D shows that a corner-shaped interstice 103 thus created receives a bony corner-shaped wedge or shim. An osteosynthesis plate is then screwed on laterally to the two sections 101, 102 to help them preserve their relative position during the period of reconstitution of the tibia 100.

FIGS. 2A and 2B give a view in two different lateral directions of the position of the osteosynthesis plate and of its screws.

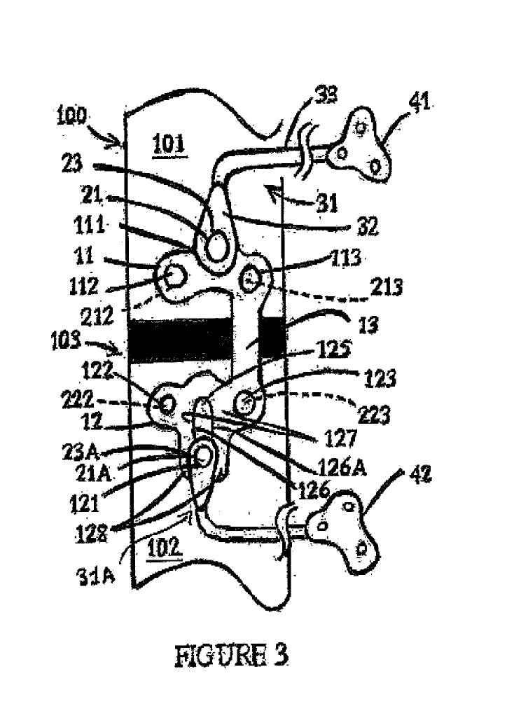

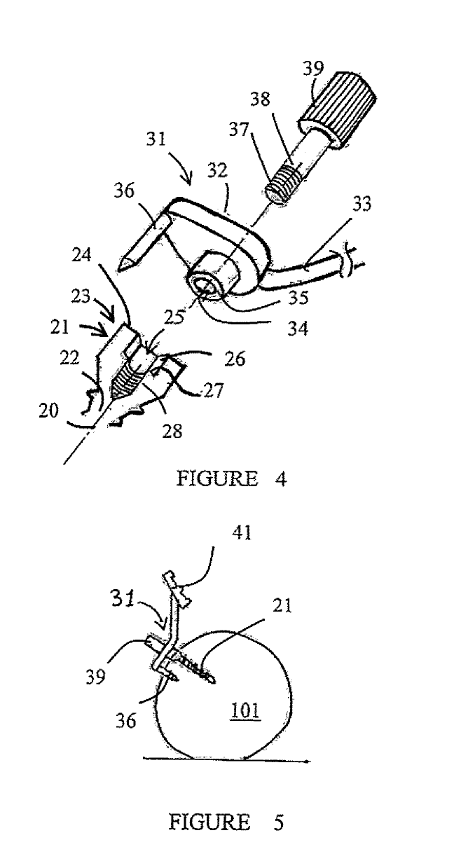

FIGS. 3, 4 and 5 show a device comprising an osteosynthesis plate in a first embodiment. The device can used to obtain positional information on the first section 101 and the second section 102 that faces the first section 101 and is separated from the corner-shaped interstice 103 in a lateral view, here a frontal view, of the patient. The osteotomy performed is designed to enable the shifting of the second section 102 and especially to re-tilt its direction of extension 150 which has proved to be a cause of problems of arthrosis in the knee. The aim is to change the tilt this lower second section 102 to bring an ankle joint center towards a reference direction defined by a hip joint center and a knee joint center. The corresponding incision and therefore the interstice 103 is just below the knee, i.e. the first section 101 is a very short upper section.

The osteosynthesis device shown fulfils two functions, the first being that of fixing first and second position-tracking devices 41, 42 and orienting first and second respective sections 101, 102 thus providing information on guidance, or navigation, enabling the surgeon to suitably adjust the angle of tilt. A slight transversal translational shift and a slight rotation of the second section 102 about its axis of direction of extension 150 are also permitted if need be. The second function is that of then blocking the second section 102 relatively to the first section 101 in the desired relative position. The final locking in position is provided by a holding element constituting an osteosynthesis plate 1 whose lateral surface is slightly rounded in a shape of a cylindrical sector to be placed flat against a rim sector of the bone 100, in going beyond it on either side of the interstice 103 to overlap two respective edge zones of the first and second sections 101 and 102. It will be noted that the osteosynthesis plate 1 does not necessarily have a uniform thickness since its cooperation with the bone 100 is limited to the two cylindrical surfaces mentioned here above. Quite clearly however, the osteosynthesis plate 1 is preferably free of any relief features which would increase its volume and therefore increase discomfort.

The osteosynthesis plate 1, which is a monoblock unit in this first embodiment, is functionally constituted by three functional zones or blocks. It comprises first and second bases 11 and 12 designed to be fixedly joined to the first and second sections 101, 102 respectively and a linking element, here in the form of a tab 13, to mutually fix the relative positions of the first and second bases 11, 12. As indicated, the osteosynthesis plate 1 is herein a monoblock unit, i.e. non-deformable, but it nevertheless enables the desired setting of the position of the second section 102 as explained here below. It can be seen that the third embodiment has an osteosynthesis plate which, on the contrary, is constituted by several distinct elements assembled adjustably and it comprises elements for mutually blocking these elements against any deformation when the second section 102 is in the desired position.

A first attachment and support screw 21 goes through a first attachment hole 111 of the first base 11 to subsequently fix the first base 11 to a said rim sector of the first section 101 and thus fixedly join the first base 11 in a fixed position on the first section 101. The first attachment and support screw 21 has another function, which is used before the first function, this other function being that of serving as a temporary base for the reception and removable attachment of a first support 31 capable of bearing the first position-tracking device 41.

For the convenience of the osteotomy, the attachment hole 111 is herein a notch on an upper edge of the first base 11, i.e. an aperture limited by two side toes 111P opened opposite an aperture 125 as indicated further below. As explained further below, the first attachment and support screw 21 is initially screwed in only incompletely so as to make it possible, after the tibia 100 has been cut, to slip in the toes 111P beneath the head 23 of this screw and screw it in completely.

The first attachment and support screw 21 has a body 22, with an axis 20, to be screwed into the first section 101, the head 23 serving as a said temporary base. In this example, the head 23 thus has an tip surface 24 comprising a relief feature for receiving and coupling the bracket 31, specifically a bore in the form of a substantially axial coupling well 25, with a side wall 26 and a ring-shaped bottom 27, in this case radial, in the position of a shoulder of a tapped attachment hole 28, with a direction called an axial direction, made in the bottom of the coupling well 25. It will be noted however, that the axial orientation of the tapped attachment hole 28 can generally be chosen independently of the axis 20, i.e. the first attachment and support screw 21 has two attachment means (body 22, head 23) which are mutually independent, their only necessary functional relationship being that their mutual positions are fixed. There is thus correspondence between the position and orientation of the first section 101 and the position and orientation of the first position-tracking device 41 which thus duplicates the position of the first section 101.

It can be understood therefore that once the second section 102 has been equipped in the same way with a second position-tracking device 42, processing means such as a computer receiving information on the position and orientation of the two position-tracking devices 41, 42 can compare them to then carry out an inverse transposition, i.e. to present the relative positions of the first and second sections 101, 102 on a screen to guide the surgeon.

The first bracket 31 has a plate-shaped base 32 carrying an arm 33 to support the first position-tracking device 41. The base plate 32 is drilled with a transversal passage hole 34 which is extended, on a side called an internal side, by a tubular coupling neck 35 with a shape complementing that of the coupling well 25. The base plate 32 herein has a pointed anchoring pin 36 extending in a direction substantially parallel to and in the sense of the direction of extension of the tubular coupling neck 35. The anchoring pin 36 can be fixed or else, as in the drawing, it may be constituted by a screw passing through a tapped hole of the base plate 32 to thus reach the first section 101, independently of the uncertainty as regards the forward progress of screwing of the first attachment and support screw 21.

The coupling neck 35 gets housed, adjustably in cross section and in length, in the coupling well 25 to thus fix a predetermined position of the first bracket 31 and therefore also of the position-tracking device 41 relatively to the first attachment and support screw 21. If the first position-tracking device 41 is not on the geometrical axis of the tapped attachment hole 28 as is the case here, it is possible to provide for a marker or index of the angular position of the base plate 32 relatively to the screw head 23. It can also be planned that the coupling well 25 and therefore the coupling neck 35 will have a non-circular cross section that allows the coupling neck 35 to be mounted only in a predetermined angular position or in a limited number of such positions. It can also be planned that the bottom ring 27 will not be in a radial plane, for example that it will be either oblique relatively to the radial or comprise error-preventing relief features such as for example a groove or a ridge.

The neck 35 is kept in a state of being pressed into a coupling position by a first position locking screw with an end threaded section 37 that gets screwed into the tapped attachment hole 28. The threaded section 37 is the extension of a “smooth” extending section 38 with a length corresponding to the depth of the coupling well 25 linked to a head 39 of the locking screw, of which a lateral rim surface herein has rough features to constitute a thumb wheel. The first bracket 31 is thus clamped between, firstly, on the side called the internal side, the well bottom radial ring 27 in contact with an end surface of a complementary shape of the coupling neck 35 and/or the tip surface 24 and, secondly, on the side known as the external side, the bottom of the locking screw head 39 which presses an external surface zone in a position of a shoulder of the passage hole 34.

FIG. 3 thus illustrates the final attaching of the bone sections 101, 102 in a side view of the patient, on the side of the interstice 103 having the maximum opening.

To ensure a more solid blocking in position on the tibia 100, the first base 11 comprises, in this example, a pair of additional attachment holes 112, 113 for the passage of a pair of respective additional attachment screws 212, 213 (FIG. 13). Each of the additional attachment screws 212, 213 may be of a classic type or else of the type of the first attachment and support screw 21 to thus implant several position-tracking devices (41) on the first section 101 or else again to support a same position-tracking device 41 on several attachment and support screws 21. In such a case, the anchoring pin is a pin of the thumb-wheel type 37-39. Since the first base 11 is fixed to the first section 101 only after the distraction operation, the additional attachment screws 212, 213 are screwed in at the end of the operation.

The second base 12 has a same pair of attachment holes 122, 123 for the passage of the additional attachment screws 222, 223 (FIG. 13) which will be screwed in only at the end of the operation. The second base 12 differs from the first base 11 in that the first attachment hole 111 is replaced therein by the aperture 125 with a longitudinal extension relatively to the direction of extension of the bone 100, which is vertical in FIG. 3. The aperture 125 has two opposite longitudinal sides 126, 126A and is herein open at its lower end, opposite the first base 11. A second attachment and support screw 21A, identical to the first screw 21 is incompletely screwed in, i.e. screwed in while leaving a depth free beneath the head 23A of the second attachment and support screw 21A. It is thus possible subsequently to slide external edge surfaces 127 of the window 125 beneath the head 23A, i.e. the second attachment and support screw 21A acts a centering pin and its head 23A provides for the final clamping in position by means of an additional tightening operation. The term “external” designates an accessible surface, i.e. a surface in a radially distal position relatively to the first section 101 which is therefore considered to occupy a “internal” position.

The external edge surface 127 is a track forming a longitudinal “safety slideway” on one of the longitudinal edges of the aperture 125, a same external edge surface 127 being possibly symmetrically planned on an opposite edge as shown in the drawing. The term “safety” indicates that the main function is that of limiting any lateral excursion relatively to the direction of longitudinal sliding, while providing for low or even zero clearance between the two facing surfaces (23A, 127), each being a “safety slideway” for the other. A permanent contact during the longitudinal sliding, i.e. mutual support, between the two facing surfaces, is permitted if it does not create an excessive friction force but this permanent contact is not necessary.

The external edge surface 127 thus has two functions of similar natures. The first function is the “safety slideway” type function, i.e. the external edge surface 127 extends in parallel to the longitudinal path of the head 23A and precisely just above it, the second section 102 of the bone 100 being positioned beneath. Thus, if the handling of the second section 102 by the surgeon causes the second attachment and support screw 21A to slide longitudinally, it can happen that the second section 102 will tend to move away from the overall plane of extension of the second base 12 constituted by the plane of the drawing of FIG. 3, i.e. it will tend to go even further behind this plane. In such a case, the underneath of the head 23A follows this motion which pushes the second attachment and support screw 21A even further into the aperture 125 so that the bottom of the head 23A thus comes immediately to “land” on the external edge surface 127 since there was only a small clearance.

The second function of the external edge surface 127 is therefore a function of a “landing strap” and “braking strap” for stopping the bottom of the head 23A, since the pressure exerted by the bottom of the head 23A, through a coefficient of friction existing between the two surfaces in contact (head 23A, surface 127), gives a component of longitudinal force that counters any subsequent longitudinal sliding. The bottom of the head 23A thus constitutes a braking pad that provides clamping.

The aperture 125 offers a template for width-wise passage corresponding to the diameter of the body of the second attachment and support screw 21A, so as to thus prevent any lateral shift of the second section 102, i.e. a leftward or rightward shift in FIG. 3. Relatively to the first base 11 and therefore relatively to the first section 101, there therefore remains only one degree of freedom of motion for the second attachment and support screw 21A, and therefore also for the second section 102, along the longitudinal direction vertical to FIG. 3. The width of the aperture 125 may however be increased if an ability to make a lateral shift of the second section 102, i.e. a shift horizontal to FIG. 3, is required.

The second attachment and support screw 21A receives a second element called a bracket, referenced 31A, bearing the second position-tracking device 42. When the desired setting of the re-tilt of the second section 102 is made and the brackets 31, 31A have been removed, the second base 12 is fixed to the second section 102 by the screwing in of additional attachment screws 222, 223 and by an additional turn of the second attachment and support screw 21A, a fraction of a turn being sufficient since the bottom of the head 23A is already almost in contact with the external edge surface 127.

As a variant, the attachment and support screw 21A has two stages, i.e. once it has been unscrewed, the head 23A receives the second bracket 31A, a positional clamping or locking screw 227 (cf. FIG. 27), the bottom of the head of this screw resting on the external edge surface 127. The head 23A then fulfils only the first function, namely the slideway function, and the second function, namely the brake function, is fulfilled by the head of the clamping screw 227.

In such a case, the aperture 125 has a cross-section profile that flares out towards its external part, hence opposite to the second section 102. The flanks 126, 126A are therefore tilted and turned partially towards the external edge surface 127. As a result, the aperture 125 offers a template of greater width in its upper part, i.e. its external part, so that the head 23A can get totally housed in the aperture 125. Thus, a tip surface (corresponding to the surface 24) of the second attachment and support screw 21A can descend until it is below the level of the external edge surface 127 so as not to hamper the setting up of the contact blocking the bottom of the head of the clamping screw 227 on the external edge surface 127. Thus, at least one inclined side 126, 126A of the aperture 125 fulfils the first function, that of the safety slideway, i.e. it receives, almost in a leaning position, an edge of the bottom of the head of the clamping screw 227 which thus serves as a safety slideway. As a variant, if the tip surface of the head 23A remains above the external edge surface 127, the head of the above thumb wheel will be planned so as to have an axial profile shaped like an overturned cup, whose free volume will constitute a housing to retract the tip of the head 23A therein. A lower edge of the cup will thus rest on the external edge surface 127.

The pinching by each toe 128 is thus effected by pressure on the two external edge surfaces 127 of the aperture 25, which are tracks overhung by the head of the clamping screw 227. It will be noted that this pinching could be limited to only one of these two external edge surfaces 127. In general, the aperture 125 which herein, in a plane view, forms an overturned U comprising two longitudinal toes 128 can be limited to a single toe 128 which is to be pinched.

In yet another variant, in the case of the aperture 125 with the flared transversal profile, an additional turn of the second attachment and support screw 21A ensures that the requisite degree of braking is set. The two said external edge surface functions 127, i.e. the functions of safety slideway and braking track are then fulfilled by at least one of the oblique sides 126, 126A. The remaining operational steps shall now be explained in detail.

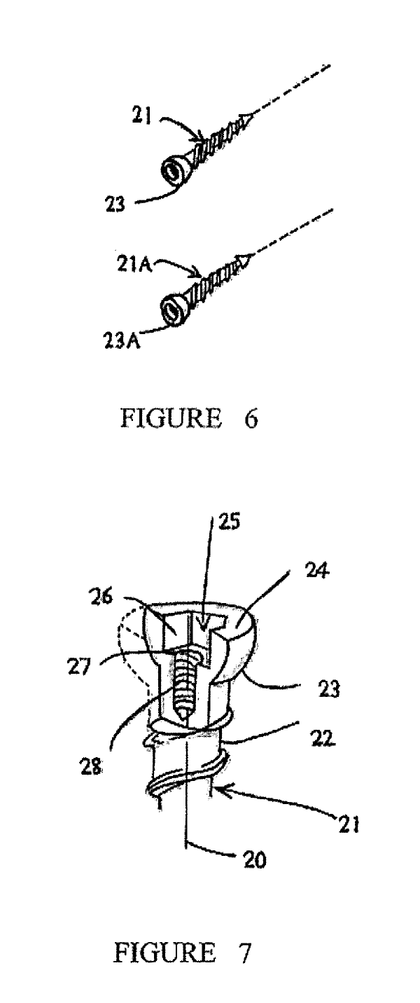

FIG. 6 shows that, in an initial step, the first attachment and support screw 21 is screwed into the first section 101 and the second attachment and support screw 21A is screwed into the second section 102. As referred to here above, the first and second attachment and support screws 21, 21A are screwed in only incompletely to enable subsequent sliding of a respective zone of the first base beneath their heads 23 and 23A.

FIG. 7 is a partially exploded side view of the head 23 showing the well 25 and the tapped hole 28.

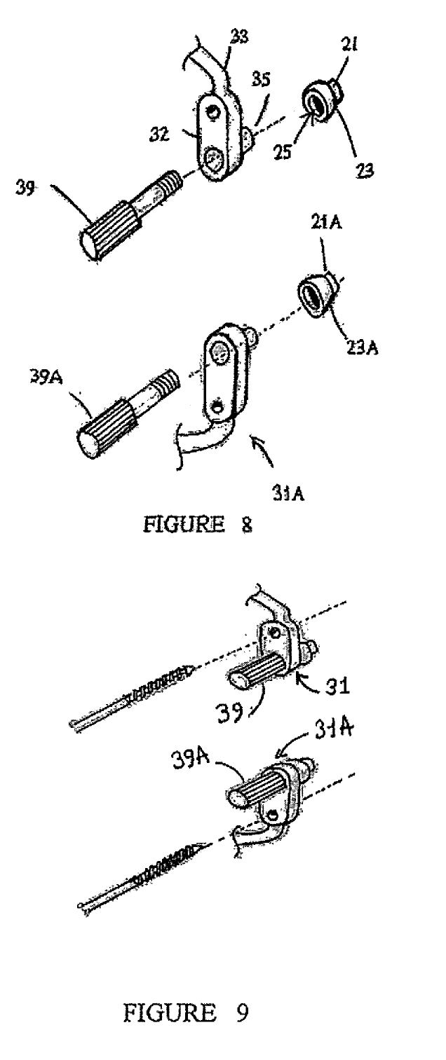

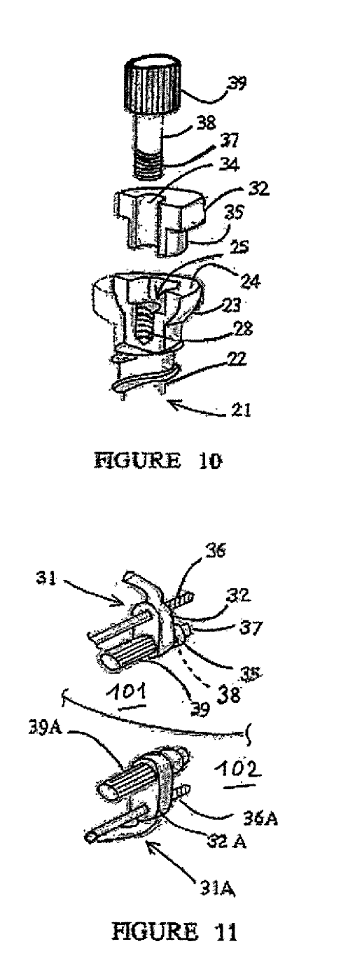

FIG. 8 is an exploded view showing the thumb wheel 37-39 as well as a homologous headed thumb wheel 39A which will respectively fix the first and second brackets 31, 31A. FIG. 9 shows that the anchoring pin in the form of a screw 36 and a homologous pin in the form of a screw 36B are then screwed in to get anchored into the associated section 101 or 102. FIG. 10 shows the thumb wheel 37-39 whose threaded section 37 will be screwed into the tapped hole of the head 23 in crossing the hole 34 of the base plate 32.

FIG. 11 shows that with the brackets 31, 31A being thus stably implanted on the respective sections 101, 102 and the position-tracking devices 41, 42 having therefore been implanted, the tibial cut is made at an intermediate axial level between the two attachment and support screws 21, 21A. Then the second section 102 is shifted up to the desired position to adjust the desired aperture and slope and the second section 102 is held in position by means of two distractors taking support respectively on the first and second sections 101, 102.

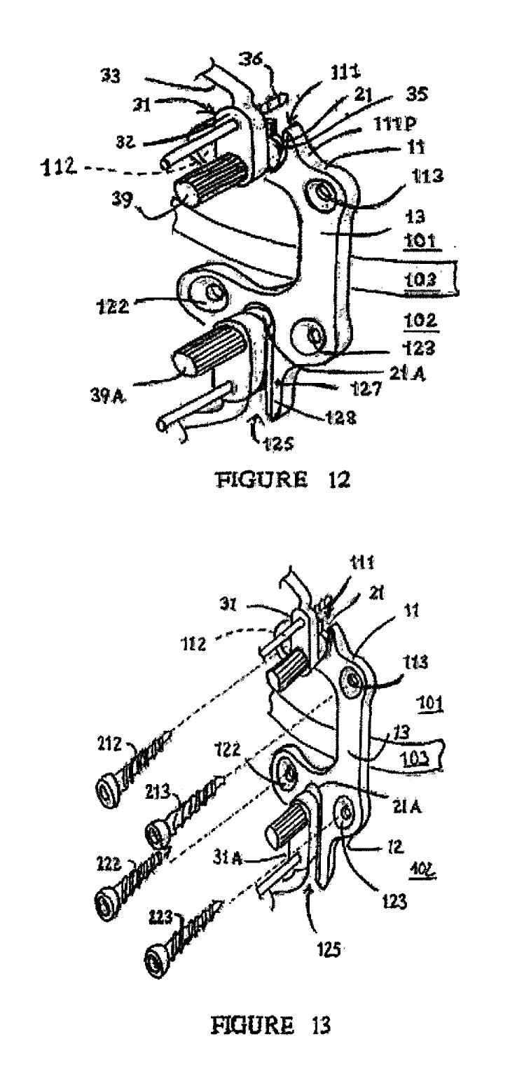

FIG. 12 shows that the osteosynthesis plate 1 is then slid beneath the heads 23, 23A. Specifically, the osteosynthesis plate is presented in an orientation tilted in its plane to slide the toes 128 of the aperture opening out 125 on either side of the axis 20, i.e. so that the head 23a overhangs the external edge surfaces 127. The aperture 125, in the form of an overturned U, is axially deep so that, when the base of the U abuts the body of the second attachment and support screw 21A, the osteosynthesis plate 1 occupies a position that is low enough for it to return to the desired purely axial orientation, i.e. without the lateral toes 111P of the first attachment hole 111 hampering this motion. The distance between the attachment and support screws 21, 21A is therefore at least equal to a value of distance between an end of one of the toes 111P (the shortest toe 111P if the toes are designed with unequal lengths, which is not the case here) and the base of the U of the aperture 125. It can be noted that it could be that the toes 111P are not parallel, i.e. that the attachment hole 111 may be a flared-out notch when seen in a plane view.

FIG. 13 shows that the two pairs of additional attachment screws 212, 213 and 222, 223 are then screwed respectively into the first and second sections 101, 102. The osteosynthesis plate is thus solidly implanted. The attachment and support screws 21, 21A are furthermore fully screwed in.

FIG. 14 shows that the brackets 31, 31A have been withdrawn by unscrewing the thumb wheels 39, 39A.

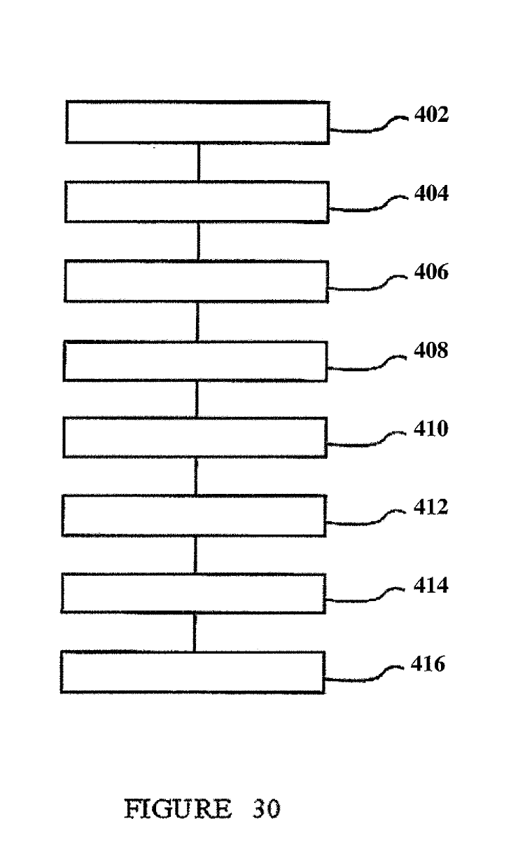

The operational procedure may then consist, with reference to FIG. 30, of the following steps:

implanting the two attachment and support screws 21, 21A in the first and second sections 101, 102 of the bone 100, step 402,

mounting the position-tracking device brackets 31, 31A bearing said devices 41, 42, step 404,

performing a bone-cutting operation, step 406,

performing, preferably by means of a distractor, an operation for distracting the first and second sections 101, 102 in keeping them in a desired final position forming an interstice, step 408,

placing an osteotomy plate 1 on the attachment and support screw 21, 21A, step 410,

carrying out a final clamping, in placing clamping screws on the head of the attachment and support screws 21, 21A or else in making an additional turn of the attachment and support screws 21, 21A, step 412,

placing spacers in the interstice thus opened, step 414,

withdrawing the distractor if any and the brackets 31, 31A, step 416.

In the other embodiments referred to here below, it will be seen that it is possible to initially mount the first and second bases because the mechanism initially leaves the desired degree or degrees of freedom between the first and second bases.

Preferably, for higher stability after the operation, the attachment screws 212, 213 and 222, 223 are placed in order to block the plate 1. The attachment screws 212, 213 and 222, 223 are then preferably screwed in before the dismantling of the brackets 31, 31A which precedes the final tightening of the first and second attachment and support screws 21, 21A.

### 2. Second Embodiment

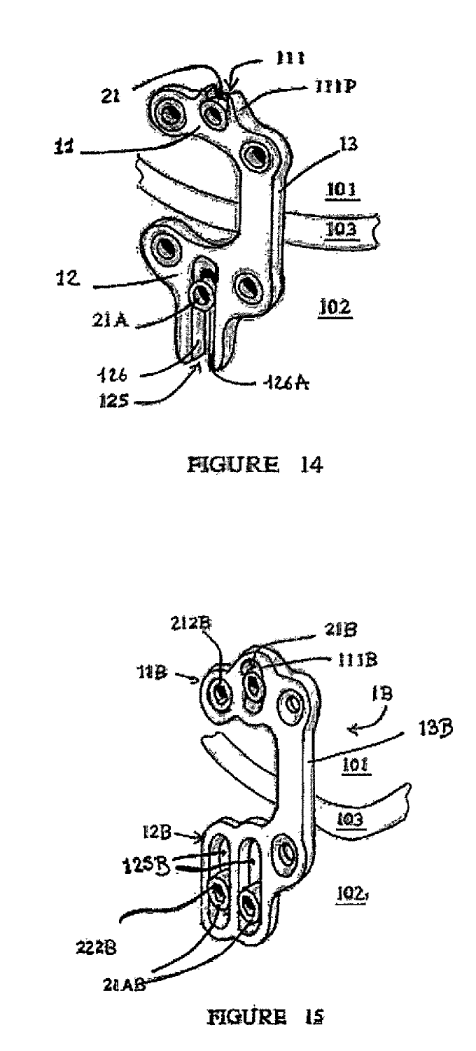

FIG. 15 is a view in perspective of the second embodiment of an osteosynthesis plate. The elements identical to those of the first embodiment have kept their reference numbers and the functionally homologous elements carry an additional suffix B.

The plate 1B of the second embodiment has an overall shape similar to that of the plate 1, with a first base 11B, a second base 12B and a linking tab 13B. However, it differs from the plate 1 in the fact that the first attachment hole 111B in the first base 11B is a hole with a closed rim, that is circular or axially oblong. The aperture 125, herein referenced 125B, is in duplicate, and forms an axially oblong hole with a closed rim. It will be noted however that the fact the rim of the first attachment hole 111B and the apertures 125 are closed does not constitute a necessary characteristic, but means only that the coupling with the attachment and support screws 21B, 21AB does not require a cut in this rim.

Specifically, the first attachment and support screw 21B and the pair of attachment and support screws 21AB herein have respective heads 23B, 23AB which are “narrow”, i.e. having a diameter that does not exceed the width of the first attachment hole 111B and of the aperture 125B. For the convenience of the explanation, the width of the first attachment hole 111B and the width of the apertures 125B are assumed to be identical so that the first and second attachment and support screws 21B, 21AB are identical. Furthermore any operational error at this level is avoided.

The remaining operational steps shall now be explained.

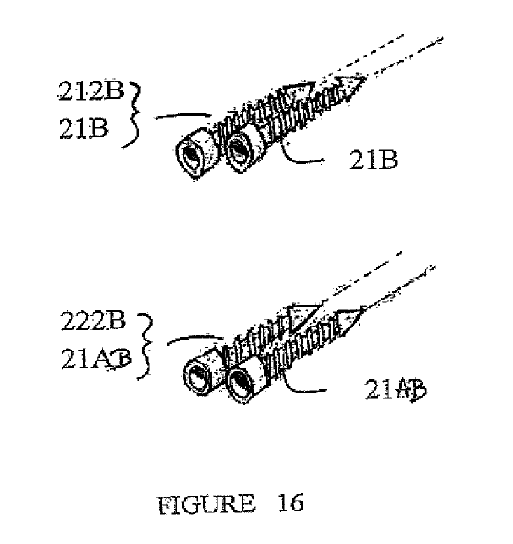

FIG. 16 shows that first of all two attachment screws are screwed into each future section 101, 102 and not only one screw as in the case of FIG. 6. Thus, the first attachment hole 111B receives the first attachment screw 21B and the attachment hole 112 receives an attachment screw 212B which is then of the type of the first attachment and support screw 21B. Similarly, the apertures 125 each receive an attachment and support screw 21AB, i.e. the attachment screw 222B is a 21A type screw.

FIG. 17 shows (in the left part) that the bracket 31 is converted into a bracket 31B in which the anchoring pin 36 is no longer a screw but a neck of the same shape as the neck 35B. The second bracket 31AB similarly has a neck 35AB and a pin 36AB. The brackets 31B, 31AB are thus each borne by two legs, hence they are more stable. The right-hand part of FIG. 17 shows the assembly position, the thumb wheels 39B, 39AB being screwed in.

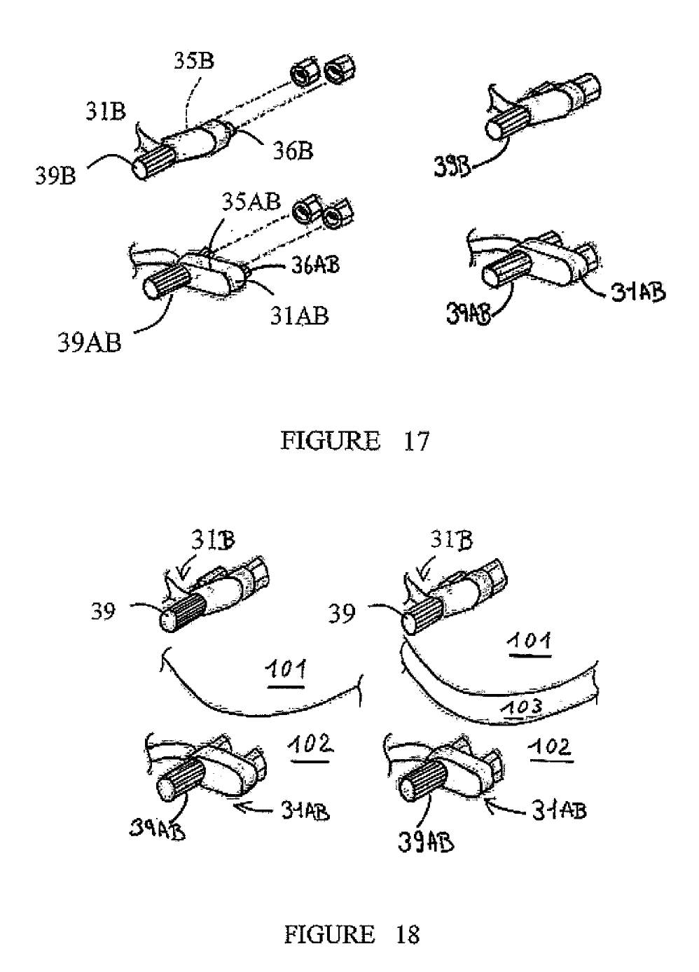

FIG. 18 shows the position of the brackets 31B, 31AB before and after separation of the first and second sections 101, 102 by osteotomy.

FIG. 15 explained here above shows that the thumb wheels 39B, 39AB have been unscrewed to remove the brackets 31B, 31AB. Since the heads 23B, 23AB of the attachment and support screws 21B, 21AB have a diameter that does not exceed the width of the attachment hole 111B and of the apertures 125B, the heads 23B, 23AB constitute pins and studs for receiving the osteosynthesis plate 1B, which may thus be directly impaled on these elements.

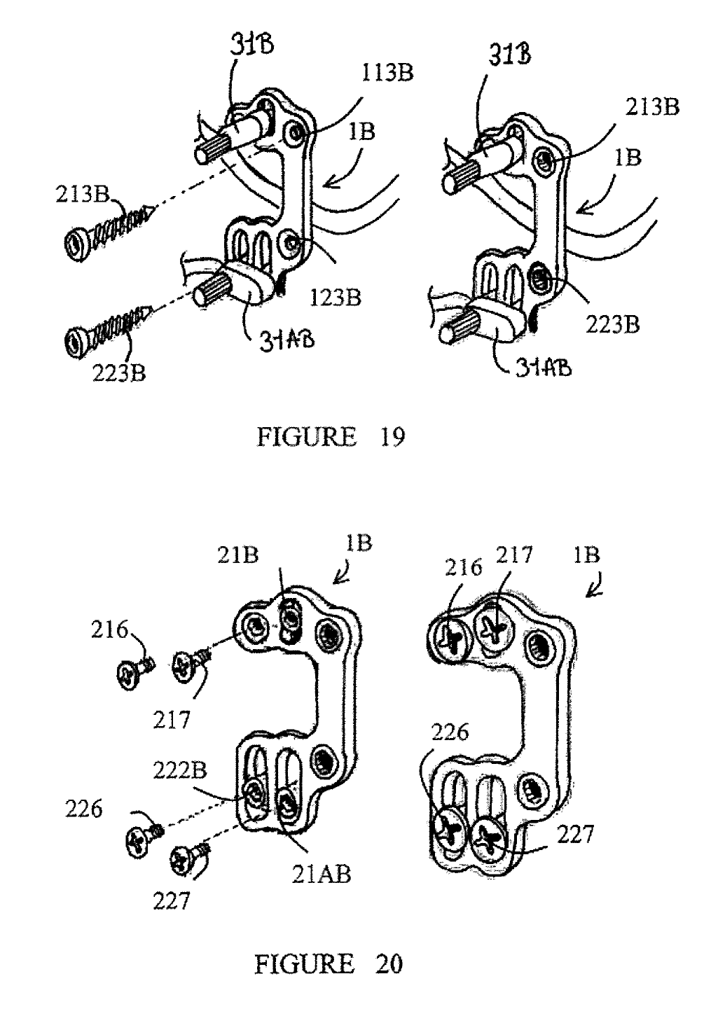

FIG. 19 shows that it is possible to reposition the brackets 31B, 31AB to verify the absence of unwanted shifting. Attachment screws 213B, 223B are also added. The left-hand part of FIG. 19 gives a detailed view of these attachment screws 213B, 223B and the right-hand part shows the state of assembly. It will be noted that the attachment screws 213B, 223B have an extended head which clamps the osteosynthesis plate 1B to the sections 101, 102.

FIG. 20 shows that the brackets 31B, 31AB have been definitively removed. To clamp the osteosynthesis plate 1B more efficiently to the first and second sections 101, 102, the tapped holes 28 of the two attachment and support screws 21B, 21AB receive respective clamping screws 216, 217 and 226, 227 whose head will fulfill the clamping function performed, in a first embodiment, by the heads 23, 23A on the toes 111P and the external surfaces 127. Since these attachment screws 216, 217 and 226, 227 get coupled with metal threads of the tapped hole 28, it can be seen that the corresponding threads can be relatively small in size, axial length and diameter since the material is resistant by nature. The size of the attachment screws 216, 217 and 226, 227 is therefore far smaller than that of the screws which would have to be implanted in the bone 100.

### 3. Third Embodiment

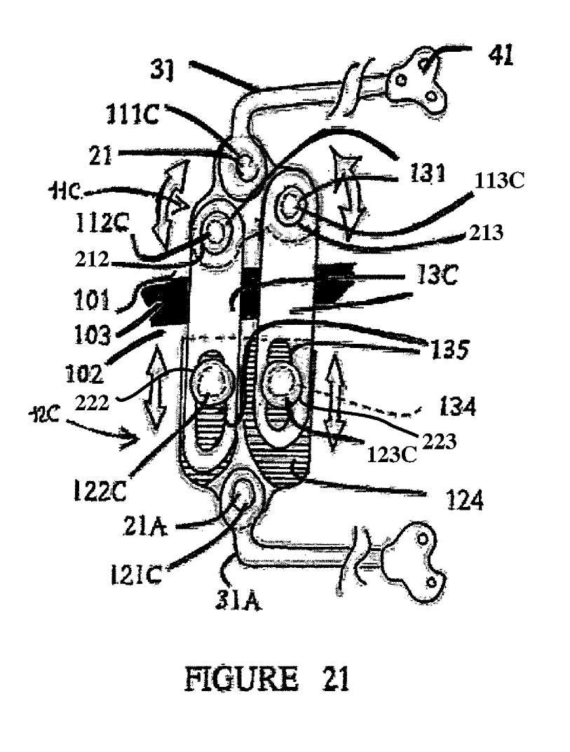

FIG. 21 is a plane view illustrating a third embodiment. The elements homologous to an element of the first embodiment bear the same numerical reference followed by the suffix C.

One original feature of the third embodiment lies in the fact that the monoblock unit formed by the three functional elements 11-13 is replaced by an assembly of three separate elements, namely a first base 11C and a second base 12C which serve as bases for an adjustable element for their mutual linkage, in the form of a strap 13C, which herein is a strap in duplicate to improve the stability and enable the setting of the slope and of the aperture.

The first base 11C is functionally identical to the first base 11 and similarly has three holes 111C, 112C, 113C, each for a classic screw or a first attachment and support screw 21. As in the case of the first base 11, the holes 111C and 113C are not aligned and, specifically, are arranged according to the vertices of a substantially equilateral triangle to have optimum bearing stability.

In this example, the first hole 111C is a tapped hole having a function identical to that of the hole 28 of the head 23. In other words, the hole 111 can be a through hole or a blind hole since its primary function then is to serve as a receptacle for the neck 35. It is therefore the first base 11C which directly bears the bracket 31.

Accessorily, it is possible however to provide for the passage into the first hole 111C, finally, i.e. after dismantling the thumb wheel 39, of an attachment screw with a body diameter smaller than that of the tapped hole so that the head of this attachment screw ensures the desired clamping by pressing on the rim area of the mouth.

The second base 12C, which is mechanically distinct from the first base 11C, similarly has three holes 121C, 122C, 123C for classic attachment screws or even possibly second attachment and support screws 21A. The hole 121C is a housing identical to the first hole 111C.

The first and second bases 11C, 12C are linked by a pair of straps 13C laid out in parallel. Each strap 13C comprises, in an upper end section, a hole 31, in this case a circular hole, for the adjusted passage of the body of the attachment screw, or even of the bracket 21, associated with the attachment hole 112C or 113C. Each strap 13C can thus occupy any angular position of a desired slope relatively to the first base 11C, as explained by the two associated curved arrows. In one lower end section, each strap 13C has an attachment hole in the form of an aperture 132 with a longitudinal extension relatively to the strap 13C, for the passage of an attachment screw, or even of a support screw 21A associated with the associated attachment hole 122C or 123C.

The presence of the apertures 135 therefore allows the fixing at the outset of the second base 12C to the second section 102 by means of the attachment screws 222, 223 since the first and second bases 11C, 12C are independent so long as the attachment screws 212, 213 and 222, 223 are not fully screwed in. In other words, the first and second bases 11C, 12C then have identical functions, allowing them to be fixed by attachment holes 112C, 113C and 122C, 123C and allowing the brackets 31, 31A to be mounted therein in the respective coupling holes 111C, 121C.

The opening required for the interstice 103 is made fixed by the tightening of the two pairs of attachment screws 212, 213 and 222, 223 which enables the application of the ends of the straps 13C to a corresponding edge surface of the associated passage hole 112C, 113C and 122C, 123C, the surfaces in contact being rough enough to have a coefficient of friction that is sufficient to prevent any shift, i.e. any deformation of the set 11C-13C. Such a deformation would correspond to a rotation of the upper ends of the straps 13C or a rotation and/or translation of the apertures 135 relatively to the second attachment and support screws 21A.

In this example, to better ensure the desired blocking of the surfaces mentioned here above, a shape matching is planned, i.e. the lower section of each strap 13C comprises, on the internal side, an undulating contact surface 134 extending in a non-longitudinal direction and, as can be seen in FIG. 21, the second base 12C has, on the external side, a contact surface 124 with undulations of complementary shapes, and having the same orientation in a coupling position. Since the direction of the undulations is transversal to the direction of extension of the aperture 135, any sliding of the aperture 135 is thus blocked. In this example, the direction of the undulations is substantially perpendicular to the direction of extension of the aperture 135. On either side of each aperture 135, on the internal surface 134, there are thus formed, on the whole, two racks of a certain width, i.e. at least one contact surface 124 or 134 has a plurality of parallel undulations thus defining grooves and a facing surface 134 or 124 comprises at least one ridge capable of being housed in any one of the grooves. It is thus possible to adjust the mutual longitudinal divergence or spacing between the first and second bases 11C, 12C while at the same time blocking any unwanted rotational motion in the plane of the FIG. 21 through the fact that at least one ridge has a certain length.

It can even be planned that a central section of the groove will be omitted, i.e. that the groove will be replaced by two mutually distant necks, used as centering pins. In the present case, the presence of the aperture 125 creates a configuration of this kind. In a similar case, the surgeon has the possibility of implanting the two pins in two separate grooves if, for any reason, he wishes to ensure that the second base 12C occupies a position with an angular orientation that is slightly modified relatively to the previous case.

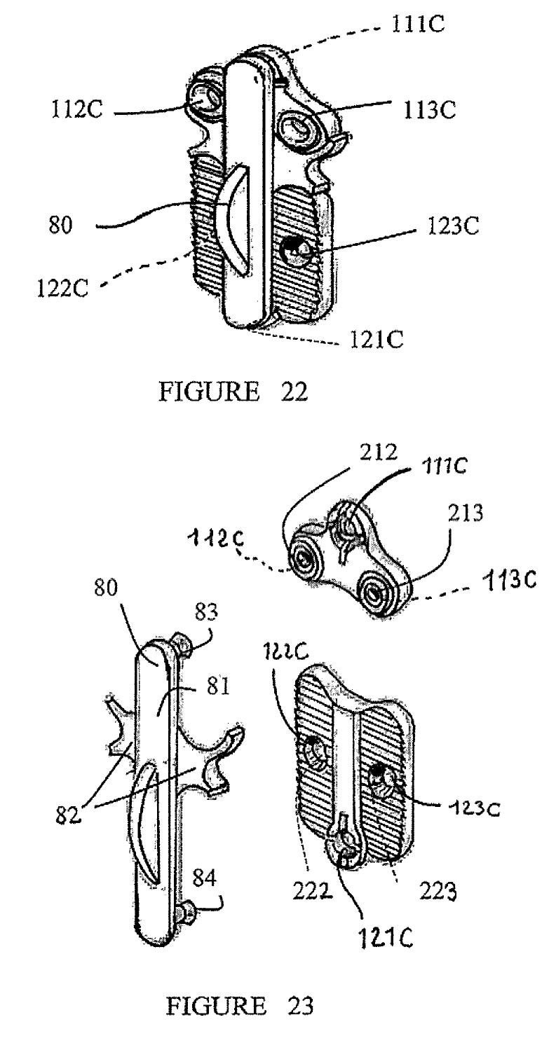

FIGS. 22 and 23 are views in perspective of the osteosynthesis plate 1C, the elements of which are borne temporarily (FIG. 22) by a grip 80 for holding the plate in functional position. The grip 80 has a trunk 81 and two opposite arms 82. Upper and lower ends of the trunk each bear a pin 83, 84 respectively housed in the first and second holes 111C, 121C. The arms 82 extend in a rear plane relatively to the trunk 81 so that the first and second bases 11C, 12C can get housed in their plane, the first base 11C being supported by a zone corresponding to a lower slice on a zone corresponding to an upper slice of the arms 82 and the second base 12C being supported, by a zone corresponding to an upper slice, on a zone corresponding to a lower slice of the arms 82. FIG. 23 shows that the grip 80 enables the application to the tibia 100 of the first and second bases 11C, 12C according to a desired longitudinal spread.

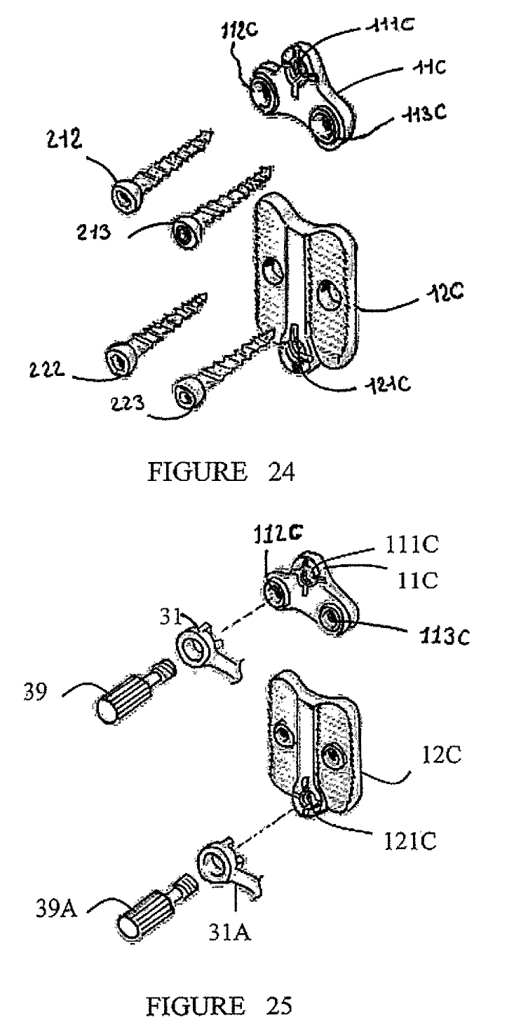

FIG. 24 shows that two attachment screws 212, 213 and 222, 223 are then screwed into the respective attachment holes 112C, 113C and 222C, 223C.

FIG. 25 is a view in perspective showing the additional elements, i.e. the brackets 31, 31A and the thumb wheels 39, 39A, to be screwed into the respective coupling holes 111C, 121C. There is therefore no attachment and support screw 21, 21A in these holes.

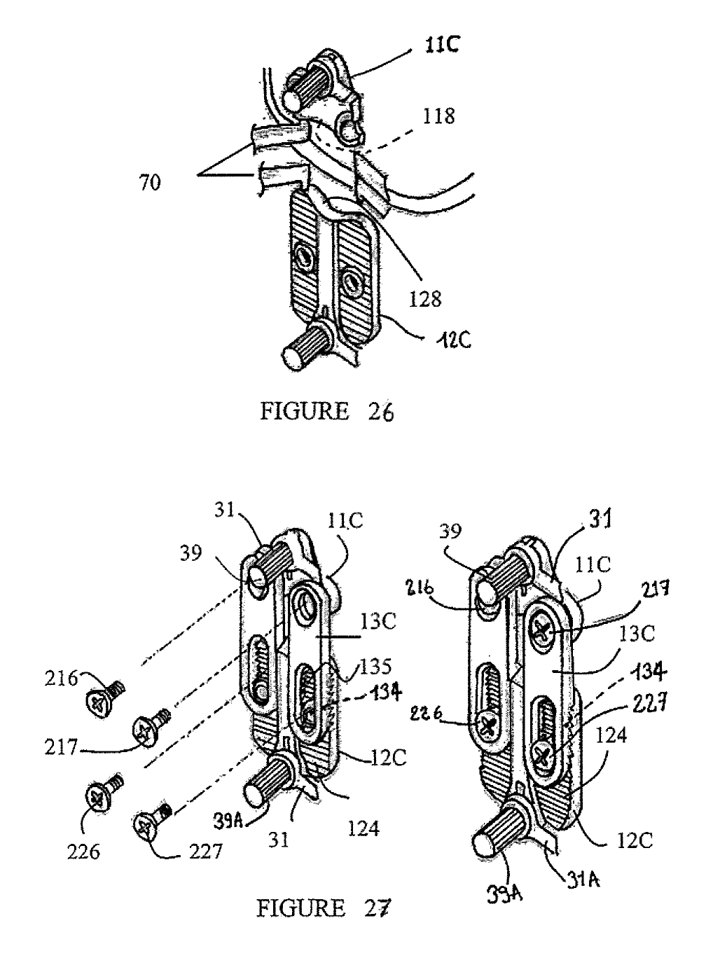

FIG. 26 shows that, once the osteotomy has been done, the sections 101, 102 are spread apart and held by two distractors 70 each of which takes support respectively on two facing zones corresponding to slices 118, 128 of the first and second bases 11C, 12C. To prevent any lateral slipping, each supporting zone corresponding to a slice has a notched profile, i.e. concave in a plane view. It can furthermore be planned that these supporting areas will be concave in the direction of thickness in the slice.

As a variant, it can be planned that each distractor 70 will be temporarily fixed, at a predetermined position, to the first and second bases 11C, 12C respectively. In other words, apart from the fact of bearing brackets 31, 31A, the first and second bases 11C, 12C are then temporarily coupled to two rigid respective arms constituting distractors. As a result, the two sections 101, 102 no longer have any degree of freedom relatively to these two arms, so that an actuator can control any motion of the distal ends of the arms so that this motion is exactly copied out at the first and second sections 101, 102. The actuator can be a device with a motion reduction mechanism to improve the final precision, this device being controlled by hand by the surgeon or through an electronic driving circuitry. In particular, an optical recognition system can provide a feedback control loop in observing the relative positions of the first and second positions 101, 102 to control the device until a relative instructed position initially given to the device is reached.

FIG. 27 illustrates the mounting of the two pairs of clamping screws 216, 217 and 226, 227 on the heads 23B, 23AB of the attaching support screws 21 and 21A in order to clamp the straps 13C.

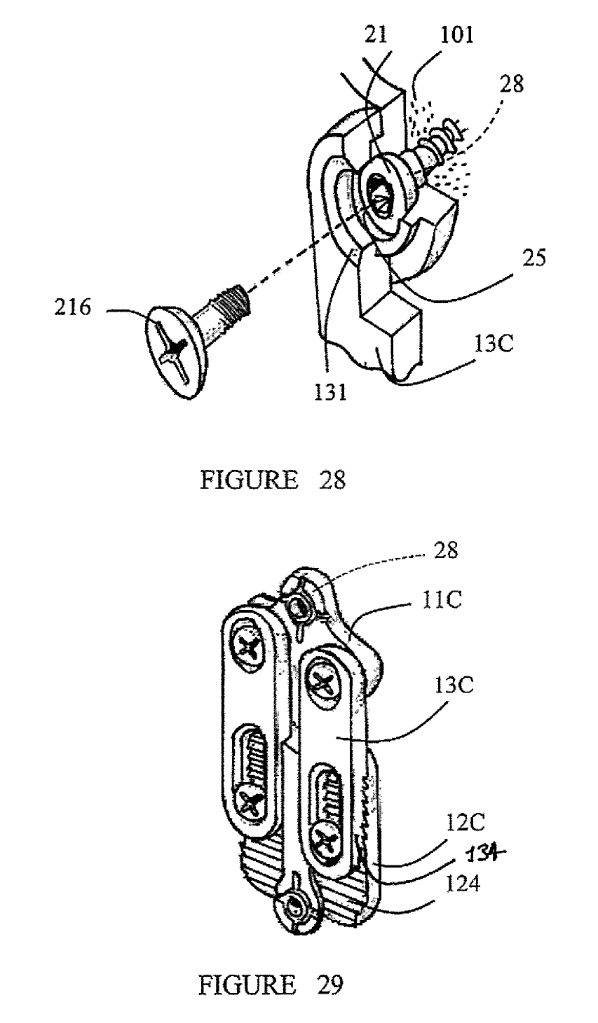

FIG. 28 is an exploded view in perspective representing the clamping screws 216 which will be received by the tapped hole 28 of the head 23.

FIG. 29 illustrates the fact that, in a final step, the brackets 31, 31A are removed and the two tapped holes 111C, and 121C are thus released by the thumb wheels 39, 39A. They may respectively receive two classic attachment screws. It will be noted that, in all the above examples, the thumb wheels 39, 39A may be omitted, i.e. that the neck 35 can include a coupling thread for coupling with the hole 28 or 111C, 121C or a clip. In this case, preferably, the neck 35 has a non-circular cross section to take up a predetermined angular position.

### 4. Other Characteristics and Variants

The above examples correspond to preferred embodiments. Consequently, certain characteristics, not vital with regard to the concept of an embodiment of the invention, appear therein routinely. In particular, all the examples have toes 126 or apertures 125, 135 whose longitudinal edge 127 or side 126 represents a track on which, at any point, the bottom of a position-locking screw head can be placed. It is thus easy adjust the desired spread for the interstice 103. However, other solutions exist. For example, in the third embodiment, it is possible to provide for a set of straps 13C for which the aperture 135 is replaced by a circular or equivalent passage hole, such as a passage hole 131, i.e. with a passage template having at least three choking points defining a circle with a diameter adjusted to that of the body of the attachment screw, therefore preventing any lateral translation of the body of the screw. Each strap 13C of the set will then offer a specific distance between centers for the two opposite passage holes.

Similarly, the above descriptions relate to embodiments in which the temporary reception base of the bracket 31 is a well 25 made in the screw head 23 for this coupling by screwing into the tapped hole 28. The well 25 thus constitutes a sheath extending the threaded hole 28 so as to more efficiently maintain the neck 35 in the desired orientation. The well 25 however is only an optional improvement since the threaded hole 28 provides this same function of holding the neck 35 in a fixed position.

According to a first aspect, other types of coupling are clearly possible, such as for example a bayonet or clip-type coupling or again a stud going through a transversal attachment hole made in the wall 26 of the well 25 or the threaded hole 28, and in the neck 35.

Similarly, a dual type coupling can be envisaged, i.e. the well 25 if any and the tapped hole 28 will then belong to the base plate 32 or to the neck 35 thus modified and the tip surface 34 will have a neck equivalent to the neck 35 described. In particular, the neck of the tip surface 34 can represent this entire tip surface 34, i.e. at least one upper section of the head 23 will constitute the coupling element. Thus, as already stated at the beginning, the rim of the head 23 can be threaded and comprise several bayonet coupling slots. In such a case, the head 23 can be reduced totally to a threaded section of any unspecified diameter relatively to that of the body 22 inasmuch as a radial extending of the head 23 is not needed for attachment to an edge of the aperture or an equivalent, for example if an attachment bolt (the equivalent of a clamping screw 216) is provided to get screwed onto the head 23.

According to a second aspect, the elements for receiving the bracket 31 which have been described as being integrated into the head 23 of the attachment and support screw 21 can be integrated into the osteosynthesis plate as described in the third embodiment where the coupling between the bracket 31 and the first base 11C is done through the hole 111C, identical to the tapped hole 28 of the head 23. An additional description of variants is then unnecessary since it suffices to state that the coupling elements of the examples here above and the various alternative embodiments can be integrated into the osteosynthesis plate, in being accessible from the external side. It can be noted moreover that, in this way, an embodiment of the invention removes the need for the angular tightened position of the screw 21, which no longer serves as a bearing element for the position-tracking device 41. It is then possible to provide for any desired orientation for receiving the bracket 31 and, furthermore, it is easy to provide for the reception in a stable fashion of a same bracket 31 resting on several points of the osteosynthesis plate and/or for receiving several brackets 31, each having any desired orientation. The position-tracking devices 41 can also be mutually spread apart so as to improve the tracking.

### 5.Fourth Embodiment, Ball-and-Socket Joint

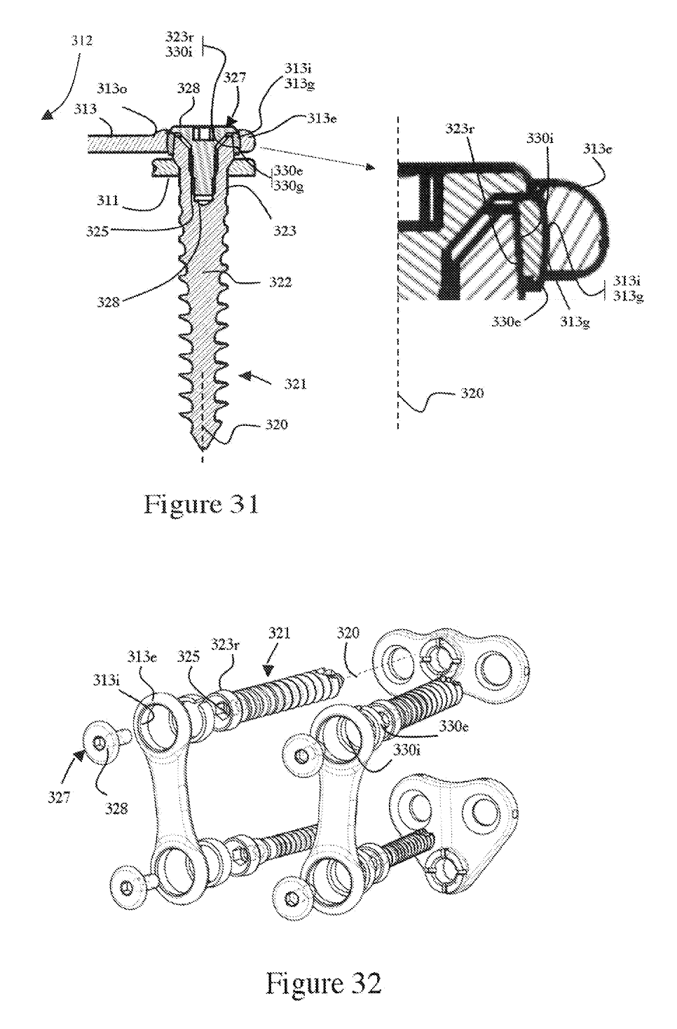

FIGS. 31 and 32 are respectively a view in axial section of another first attachment and support screw, herein referenced 321 and an exploded view of the device, the references of the various homologous elements of the previous figures have been kept except that all the references are given in the 300s; The first attachment and support screw 321, with an axis 320, has a body 322 with a head 323, the bottom of which is used to clamp the first base 311 to the bone 100. One end of the tab 313, providing a bridging link with the second base 312, forms a slot 313o designed to extend substantially in a radial plane relatively to the axis 320 but, all the same, with a possibility of adjusting its orientation along three independent axes of rotation. The slot 313o offers a chamber forming a circular passage 313p with an axis 320 whose lateral wall is a portion of a concave spherical surface defining a housing volume template 313g.

The housing template 313g has a center of sphericity herein situated on the axis 320, it being understood however that it could be elsewhere. Similarly, this center of sphericity is herein situated in a slice of space demarcated by the top and bottom of the slot 312o. However, this center of sphericity could be designed to be outside this section and for example above the slot 313o, i.e. beside the first base 311, the shape of the concave internal surface 313i thus tending to be that of a cap.

As above, the head 323 has a coupling well 325 comprising a tapped attachment hole 328 receiving a short clamping screw 327 whose clamping head 328 has a non-circular tip cavity, in this case a hexagonal cavity, to be screwed in or to receive said bracket 31. The clamping head 328 forms a radially spreading fold having a template of radial space requirement of a value greater than that of a homologous template of radial space requirement template for the head 323. Thus, in FIG. 31, where the axis 320 is vertical, the clamping head 328 overhangs a clamping ring 320 housed between the concave internal surface of the slot 313o and a lateral surface 323r of the screw head 323.

The clamping ring 330 thus has a convex external surface 330e defining a template of volume space requirement 330g with a sphericity corresponding to that of the housing template 313g of the concave internal surface 313i of the slot 313o and presents an internal surface 330i of axial sliding on the lateral surface 323r. The two mutual axial sliding surfaces 330i and 323r may or may not be of circular radial section, i.e. the clamping ring 330 may or may not rotate on the first screw 321. At least one of the axial sliding surfaces 330i and 323r is tilted relatively to the axis 320 to thus form a ramp for the external, radial re-tilting of the trajectory of each of the elementary constituent units of the clamping ring 330. In other words, the axial downward thrust of the clamping ring 330, under the effect of the screwing in of the clamping screw 327, causes the clamping ring 330 to widen, i.e. causes an increase in its diameter. To this end, the clamping ring 330 can be deformed elastically or plastically because no pull-back effect is expected.

It may be planned that the clamping ring 330 will split crosswise forming a partial ring, if its constituent material has excessive resistance to deformation in expansion of its diameter. As a variant, the clamping ring 330 is split slantwise essentially lengthwise, i.e. it is constituted by a helical winding of turns of a wire initially formed to comply with the desired template of overall space requirement 330g. The turns can thus slide tangentially to each other in order to partially unroll and thus have their diameter increased.

The increase thus prompted of the radial space requirement of the template of volume space requirement 330g of the clamping ring 330, hence its spherical radius, leads the external surface 330e to firmly press on the internal ring 313i of the slot 313o. This results in blocking any mutual rotation when the tab 313 is considered to be in the desired direction. The template of overall space requirement 330g and housing template 313g are then identical.

However, if the sphericity values of the two templates 330g and 313g are not totally identical, the mutual contact will be limited to a single circle or to two remote circles depending on whether radius of the template of overall space requirement 330g is respectively smaller than or greater than the radius of the housing template 313g.

Even if the templates 330g and 313g have an unwanted deformation at the single circle above, their contact can be limited to three points or zones of a theoretical circle, which again ensures efficient mutual anchoring against any rotation. Indeed, the localized pressure force will prompt a latent force of frictional resistance, even an imprint, i.e. an anchoring relief feature and furthermore the lever arm of the resistance torque remains equal to the diameter of the template 330g or 313g. In an extreme case, the template of overall space requirement 330g being for example slightly ovoid, there will be no contact except at two diametrically opposite zones. Here again, the coefficient of friction between the surfaces in contact offers a certain resistance of locking in position.

The initial freedom of rotation of the spherical surfaces 330e and 330i facing each other thus offers three possible degrees of freedom in rotation, hence every possibility of setting mutual orientations of the first and second bases 311, 312, the second base 312 being herein “represented” by the tab 313. It can be seen that, although the above ball-and-socket joint offers the three possible degrees of freedom in rotation, it can however be planned that the opposite end of the tab will be designed similarly with a ball element for linkage with the second base 312. In such a case, this makes it possible to increase the angular ranges of setting between the first and second bases 311, 312. It can however be planned that one of the ball elements will deal with only two degrees of freedom in rotation and that the other will deal with the third degree of freedom, for example the rotation about the axis 320. Furthermore, it may be planned to have a set of straps 313 of various lengths.

In this example, the ramp 323r is made on a section of the first screw 321 which comprises the coupling well 325. The wall of the coupling well 325 can be split axially so that this section forms axial tabs having an elasticity in internal radial deflection so as to thus store an energy of exercise of a centripetal radial reaction force on the clamping ring 330, thus ensuring that efficient contact is maintained between the convex external surface 330e and the concave internal surface 313i of the slot 313o.

In dual fashion, it can be planned that the coupling of the two spherical surfaces 333e and 313i will be done for a centripetal radial crushing of the slot 313o, hence towards the axis 320. In such a case, the slot 313o has an upper zone, on the external side of the device, which forms a raised shoulder relatively to the tab 313 to permit the rotation of a substantially axial upper pawl borne on the periphery of the clamping head 328 of the clamping screw 327 and sliding rotationally on a radially external surface 313e of the slot 313o. The clamping head 328 thus comprehensively has the shape of an umbrella with one or more such angularly distributed pawls. According to the principle of the corner effect explained further above, the above-mentioned pawl extends slightly obliquely, radially outwards, to constitute a sort of upper flank of a thread gradually pushing back the external surface 313e, during its rotation in descent with an internal radially crushing component, therefore correspondingly diminishing the housing template 313g.

As a variant, in a dual manner, the upper part of the external surface 313e of the slot 313o is, as referred to in the drawing, having the very general form of a truncated cone in widening downwards towards the first base 311 to constitute a stop ramp for the free end of the above pawl during its axial descent. The angle of the ramp is chosen to be small enough, given the coefficient of mutual friction for the above pawl to exert, on the ramp, a centripetal radial force of deformation of the slot 313o which is appreciably amplified relatively to the thrust axial force exerted by the screwing operation.

In the case represented, the clamping screw 327 serves both to exert the axial force pushing the slider constituted by the clamping ring 330 and also serves to maintain it in the desired final position. As a variant, two distinct elements may be provided for these two functions. It is possible for example to omit the clamping screw 327, the clamping ring 330 being then “self-mobile” in axial translation in the form of a tapped nut engaged with a threading of the external surface 232e. The clamping ring 330 then has an accessible surface for driving in a screwing operation, for example an upper section of the internal surface 330i or a section corresponding to an upper slice comprising a ring of rotationally driving relief patterns such as notches. The holding of this assembly in its final position can be ensured by a frictional force of the thread surfaces in contact. However, a pin or clip is not to be ruled out for this purpose.

As a variant, relatively to this last variant, the clamping ring 330 is replaced by a ring that can be non-deformable but axially mobile and functionally associated with a fixed element, a ring or the like, for the axial holding of the slot 313o so that the housing template 313g remains in a fixed position. The center of sphericity of the concave internal surface 313i of the slot 313o will be situated in the housing template 313g so that the spherical volume thus demarcated has, in axial section, an overall shape of a rectangle whose length extends radially and whose two sides along its width are C-shaped lateral walls with facing apertures. The clamping ring at rest and the retaining element will then have external diameters or transversal templates sufficient to be imprisoned by the spherical volume thus demarcated. The mobile ring slides axially on the head 323, for example by screwing as here above, and may thus move away axially from the holding element, until it abuts a zone of the internal surface 313i close to a horn of the “C”. It is therefore a locking by lateral translation of the above ring relatively to the radial plane of a resting position and no longer a locking by expansion of its diameter.

It must be noted that there is nothing to prevent the use of various assemblies according to an embodiment of the invention for purposes other than surgical.

Although the present disclosure has been described with reference to one or more examples, workers skilled in the art will recognize that changes may be made in form and detail without departing from the scope of the disclosure and/or the appended claims.

1. An osteosynthesis device comprising:
first attachment means for attaching a first holding base to a first section of a bone,
second attachment means for attaching a second holding base to a second section of said bone, and
rigid linking means disposed so as to mutually connect the first and second bases,
wherein at least one of the first attachment means and the first base comprises first means for receiving at least one first bracket of an associated position-tracking device.

2. The osteosynthesis device according to claim 1, wherein the second attachment means comprise second means for receiving at least one second bracket of a second position-tracking device.

3. The osteosynthesis device according to claim 1, wherein at least the first receiving means are disposed so that the bracket of the associated position-tracking device is carried removably.

4. The osteosynthesis device according to claim 1, wherein the first attachment means include a first headed attachment screw comprising first receiving means disposed so as to receive and get coupled to the bracket the first position-tracking device.

5. The osteosynthesis device according to claim 4, wherein the first receiving means constitute a tip relief feature of the head of the first attachment screw.

6. The osteosynthesis device according to claim 5, wherein the tip relief feature is disposed to receive a clamping screw.

7. The osteosynthesis device according to claim 1, wherein the first bracket belongs to the osteosynthesis device and comprises a point of anchoring to the associated bone section.

8. The osteosynthesis device according to claim 1, wherein a said base comprises a toe disposed so as to slide between a surface zone of the associated bone section and an lower surface, in an overhanging position, belonging to an attaching element.

9. The osteosynthesis device according to claim 1, wherein a said base comprises an aperture for setting the position of the associated attachment means.

10. The osteosynthesis device according to claim 1, wherein the linking means comprise a strap comprising first and second coupling means for coupling respectively with the first and second bases, the first coupling elements offering a degree of freedom in rotation between the first base and the strap to set an angular value of relative rotation between the first and second bases, and therefore also of the bone sections, and the second coupling means offering a degree of freedom in longitudinal translation to set a value of longitudinal distance between the first and second bases.

11. The osteosynthesis device according to claim 10, wherein the second base comprises a rough coupling surface to cooperate with a homologous surface of the second coupling elements of the strap, in which a longitudinal aperture is made for the passage of a second headed attachment screw designed to mutually immobilize said surfaces by mutual pressure.

12. The osteosynthesis device according to claim 1, wherein the first and second bases are separate elements comprising supporting surfaces for respective distractors.

13. The osteosynthesis device according to claim 1, wherein the first and second bases are separate elements disposed so as to be mounted on a grip for maintaining relative position.

14. The osteosynthesis device according to claim 1, comprising ball-and-socket means,
comprising a pair of first and second cooperating joint means, any one of these means being associated with the first base and the other one being associated with the linking means,
the first joint means having a volume template of overall space requirement smaller than a housing volume template, presented by the second joint means, so as to be housed therein with clearance and a possibility of mutual rotation along three axes, in a state of rest of the ball-and-socket means,
and comprising locking means disposed so as to make the joint means pass into a locked state by modification of one of said volume templates so as to eliminate said clearance in at least two distinct zones of each of the volume templates.

15. The osteosynthesis device according to claim 14, wherein the template of overall space requirement is a slice of a substantially spherical volume having a first determined radius and the housing template is a slice of a substantially spherical volume having a second determined radius, the locking means being disposed to deform one of the joint means so as to at least locally modify the radius until equality is attained with the radius of the other of the joint means to thus set up a contact bringing about a passage to the locked state.

16. The osteosynthesis device according to claim 15, wherein the first joint means comprise a ring, with a radially external surface defining said template of overall space requirement, and a radially internal surface, mounted so as to be sliding on a sloping surface of an expansion neck, that is functionally a truncated cone, belonging to the locking means, this ring being associated with an element for thrusting in said sliding operation and holding in a final locking position.

17. The osteosynthesis device according to claim 16, wherein the expansion neck is constituted by a head of a -first attachment screw of the first base.

18. The osteosynthesis device according to claim 16, wherein the thrusting and holding element is a screw comprising said first means for receiving at least one said first bracket of an associated position-tracking device.

19. The osteosynthesis device according to claim 14, wherein the locking means comprise a slider laid out so as to slide in an interstice volume offered by said clearance, until the slider comes into a position of being supported on the first joint means and the second joint means.

20. The osteosynthesis device according to claim 14, wherein the template of overall space requirement offers a spherical portion and the housing template offers an associated truncated-cone portion.

21. The osteosynthesis device according to claim 14, wherein the template of overall space requirement of the first joint means is compatible with a first housing template presented by a first section of said housing belonging to the second joint means and is incompatible with a second housing template presented by a second housing section, and the locking means are disposed so as to push back the first joint means, from the first section to the second section.

22. An operating method comprising:
using an osteosynthesis device comprising:
a first attachment element for attaching a first holding base to a first section of a bone, wherein at least one of the first attachment element and the first base comprises first means for receiving at least one first bracket of an associated position-tracking device,
a second attachment element for attaching a second holding base to a second section of said bone, and
a rigid link disposed so as to mutually connect the first and second bases,
implanting the two attachment elements in the first and second sections of the bone,
mounting the at least one first brackets bearing said position-tracking device,
performing a bone-cutting operation,
performing an operation distracting the first and second sections in keeping them in a desired final position forming an interstice,
carrying out a clamping of the osteotomy plate on the first and second attachment elements.
